(19) United States
(12) Patent Application Publication
(10) Pub. No.: US 2022/0073855 A1
(43) Pub. Date: Mar. 10, 2022

(54) Cartridge

(71) Applicant: WATERSCOPE LIMITED, Impington, Cambridge (GB)

(72) Inventors: Alexander Patto, Impington, Cambridge (GB); Nalin Patel, Impington, Cambridge (GB); Sammy Mahdi, Impington, Cambridge (GB); Tianheng Zhao, Impington, Cambridge (GB); Richard Bowman, Impington, Cambridge (GB)

(21) Appl. No.: 17/309,827

(22) PCT Filed: Dec. 19, 2019
(86) PCT No.: PCT/GB2019/053621

§ 371 Date: Jun. 21, 2021

(30) Foreign Application Priority Data
Dec. 21, 2018 (GB) 1821062.5

(51) Int. Cl. C12M 3/00; G01N 33/52; C12Q 1/04; C12M 1/26; B01L 3/00; B01L 9/00; C12M 1/34

(52) U.S. Cl. CPC C12M 23/42; B01L 2200/04; C12Q 1/04; C12M 33/14; B01L 3/5023; B01L 9/52; B01L 3/523; C12M 41/36; B01L 2200/141; B01L 2300/0609; B01L 2300/0835; B01L 2300/069; B01L 2200/025; B01L 2200/0689; G01N 33/521

(57) ABSTRACT

A cartridge comprising a cartridge body and a capture medium holder disposed within the body, the capture medium holder comprising a platform configured to receive a capture medium, the capture medium holder being configured to move relative to the cartridge body between a sample collection position in which the platform is aligned with a sample inlet of the cartridge body and an inspection position in which the platform is aligned with an inspection window of the cartridge body.

### BACKGROUND

Embodiments of the present disclosure relate to a cartridge for use in analysis of liquids such as water or animal or human bodily fluids, and analysis of liquids using the cartridge.

Use of optical techniques, e.g. use of microscopes, in the testing of liquids to detect the presence and/or concentration of a substance or a live microorganism is long established.

However, the handling of a sample during one or more of collection, culturing and analysis of the sample may result in contamination unless conducted in a sterile environment. Transportation of a sealed sample to a sterile laboratory for testing may be both time-consuming and costly, which may be particularly problematic in an area remote from a suitable laboratory; if there is an urgent need to diagnose a disease; or if there is a need to quickly and/or cheaply determine the presence of any potentially harmful microorganisms in a liquid source, for example, a water source.

US 2014/0342774 discloses a cell culturing device including a housing that contains a lid having an optically clear window; a fluid distribution channel; a sample injection port fluidically connected to the fluid distribution channel; a base housing a porous media pad; and a media injection port fluidically connected to the media pad.

US 2002/0127630 discloses an integrated filtration and detection device for collecting and detecting the growth of microorganisms in a specimen. The device has a filter between an inlet and an outlet and a sensor in a chamber.

### SUMMARY

A summary of aspects of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects and/or a combination of aspects that may not be set forth.

The present inventors have developed a cartridge which may allow for accurate determination of the presence and/or concentration of substances or live microorganisms in a sample liquid without exposing the sample to potential external contaminants during collection, culturing and analysis of the liquid sample.

The cartridge may be used in the field, i.e. without the need for a sterile laboratory environment, and/or without significant user training to avoid contamination of a sample.

Accordingly, in some embodiments there is provided a cartridge comprising a cartridge body and a capture medium holder disposed within the body, the capture medium holder comprising a capture surface configured to receive a capture medium, and the capture medium holder being configured to move relative to the cartridge body between a sample collection position in which the platform is aligned with a sample inlet of the cartridge body and an inspection position in which the platform is aligned with an inspection window of the cartridge body.

In some embodiments, there is provided a kit for forming a cartridge as described herein, the kit comprising a cartridge body top section comprising the sample inlet and the inspection window; a cartridge body base configured to form the cartridge body with the cartridge body top section; and the capture medium holder.

In some embodiments, there is provided a method of analysing a liquid sample comprising:

introducing the liquid sample into the sample inlet of the cartridge as described herein in which the capture medium holder is in the sample collection position;

filtering the liquid through a capture medium supported on the platform to collect a sample on, or in, the capture medium;

moving the capture medium holder to the inspection position; and

analysing the collected sample through the inspection window.

In some embodiments there is provided a cartridge comprising a cartridge body and a membrane holder comprising a platform configured to receive a porous membrane disposed within the body, the membrane holder being configured to move relative to the cartridge body between a sample collection position in which the platform is aligned with a sample inlet of the cartridge body and an inspection position in which the platform is aligned with an inspection window of the cartridge body.

### DESCRIPTION OF THE DRAWINGS

Embodiments of the present disclosure are described in more detail with reference to the drawings, which are not drawn to any scale:

FIG. 1A illustrates schematically an exploded view of a cartridge according to some embodiments in which the capture medium holder is in a sample collection position;

FIG. 1B illustrates schematically an exploded view of a cartridge according to some embodiments in which the capture medium holder is in a sample inspection position;

FIG. 2A illustrates schematically a side view of a base section of a cartridge according to some embodiments;

FIG. 2B illustrates schematically a top view of the base section of FIG. 2A;

FIG. 2C illustrates schematically a bottom view of the base section of FIG. 2A;

FIG. 2D illustrates schematically a vertical cross-section through the collection region of the base section of FIG. 2A along line A-A;

FIG. 2E illustrates schematically a vertical cross-section through the inspection region of the base section of FIG. 2A along line B-B;

FIG. 3A illustrates schematically a side view of a top section of a cartridge according to some embodiments;

FIG. 3B illustrates schematically a top view of the base section of FIG. 3A;

FIG. 3C illustrates schematically a bottom view of the base section of FIG. 3A;

FIG. 4A illustrates a top view of a capture medium holder according to some embodiments;

FIG. 4B illustrates a side view of the capture medium holder of FIG. 4A; and

FIG. 4C illustrates a vertical cross-section through line C-C of the capture medium holder of FIG. 4A.

FIG. 5A illustrates an exploded perspective view of an ampoule.

FIG. 5B illustrates a schematic side view of parts of the ampoule shown in FIG. 5A.

FIG. 5C illustrates a cross-sectional view of parts of the ampoule shown in FIG. 5A.

### DETAILED DESCRIPTION

The description describes the technology in detail with reference to specific embodiments however it will be appreciated that variations of specific features and combinations of specific features described herein will be apparent to the skilled person.

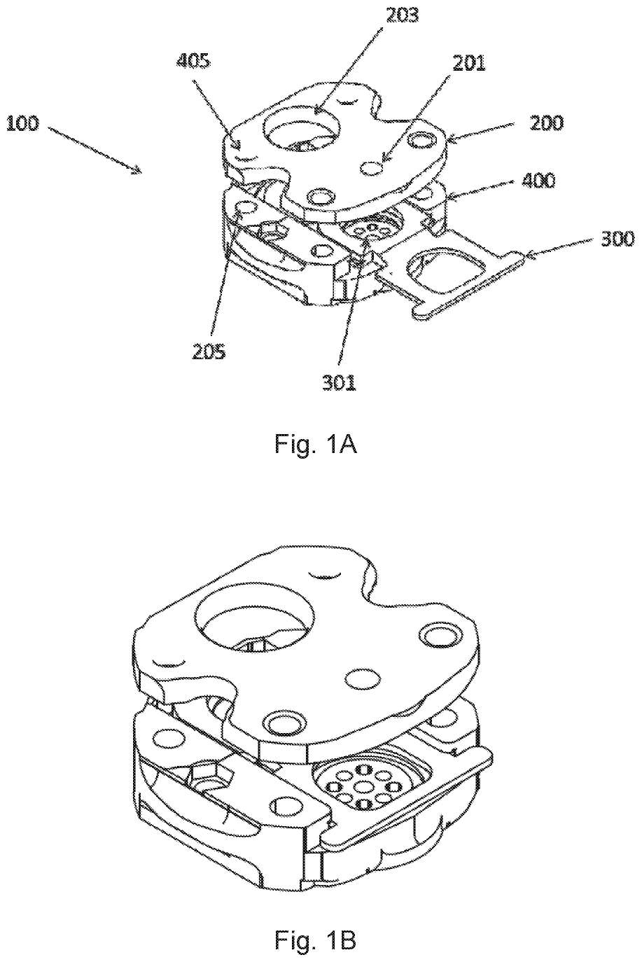

FIGS. 1A and 1B illustrate exploded views of a cartridge 100 according to some embodiments.

The cartridge comprises a body and a capture medium holder 300. The capture medium holder is engaged with and movable relative to the body. The capture medium holder comprises a platform which, when the cartridge is in use, retains a capture medium. The capture medium may be any suitable capture medium, for example a porous membrane such as a filter paper or a growth medium for microorganism growth which may or may not be porous (no capture medium is present in the cartridge illustrated in FIGS. 1A and 1B). An exemplary growth medium is an agar-based solid growth medium.

It will be understood that a “porous capture medium” as used herein means a capture medium through which a liquid sample can be filtered.

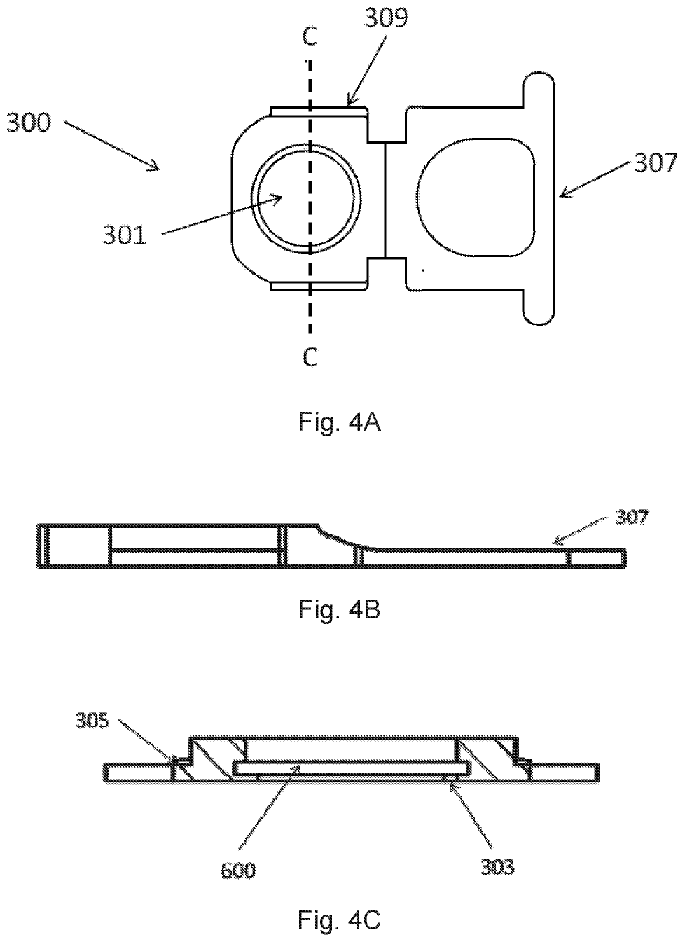

In some embodiments, the platform defines a perimeter of an aperture, e.g. a described in more detail with reference to FIGS. 4A-4C. In these embodiments, in use the platform may support a porous capture medium through which a sample liquid may be filtered, e.g. for analysis of a liquid sample having a volume which cannot be entirely absorbed by the capture medium. Excess liquid may be drained through the aperture when the capture medium holder is in the sample collection position.

In some embodiments, an analytical substance may be introduced to the capture medium through the aperture when the capture medium holder is in the inspection position.

In some embodiments, the platform is a recessed surface of the capture medium holder for retaining a growth medium. In these embodiments, the platform may or may not comprise an aperture. In some embodiments, the platform is a flat surface of the capture medium holder, i.e. a non-recessed or flush surface. A platform according to these embodiments may be used for analysis of a liquid sample having a volume which can be absorbed by a porous or non-porous capture medium and/or for which introduction of an analytical substance is not required following capture of the sample.

The cartridge body may comprise a top section 200 and a base section 400 which, when engaged together, are in direct contact and form a cavity in which the platform of the capture medium holder is disposed, the cavity having an upper cavity surface defined by a lower surface of the top section of the cartridge body and a lower cavity surface defined by an upper surface of the base section of the cartridge body. Optionally, at least part of a perimeter of the top section contacts at least part of a perimeter of the base section when the top section and base section are engaged together. The top and base sections may be disengaged such that they are not in direct contact.

The top and base sections may be engaged together by any suitable securing means, e.g. screws or bolts for use in apertures 205 and 405; one or more clamps; one or more cams; or one or more springs. The securing means may be adjustable. In a first configuration of the securing means, e.g. with the top and base sections tightly held together, e.g. tightly screwed or bolted together, the top section and base section cannot move relative to one another. In a second configuration of the securing means, e.g. with the top and base sections loosely screwed or bolted together, the top section and base section are moveable relative to one another.

In some embodiments, the securing means is the only means by which movement of the top section and base section is limited. In other embodiments, a flexible curtain (not shown) may be joined to the base section and the top section. The flexible curtain may surround at least part of the capture medium holder. The flexible curtain may allow the top and base sections to disengage from one another and move a limited distance apart from one another, the distance being limited by the height of the curtain between the base section and the top section. The curtain is preferably water impermeable. The curtain may or may not be elastic.

The cartridge body has a collection region and an inspection region and the capture medium holder is movable between a collection position and an inspection position. The cartridge may be configured to allow movement of the capture medium holder between the collection position and inspection position without exposure of a sample collected on the capture medium to an external environment and/or handling of the capture medium carrying the sample.

In the embodiment of FIGS. 1A and 1B, the cartridge is configured to allow lateral movement of the capture medium holder between a collection position and an inspection position, however it will be appreciated that in other embodiments the cartridge may be configured to allow the capture medium holder to move in a different way relative to the cartridge body, e.g. by rotation.

In the collection position, as illustrated in FIG. 1A, the platform of the capture medium holder 300 is aligned with an inlet port 201 of the top section through which a liquid sample may be introduced into the cartridge body and onto a capture medium (for example, a porous membrane, such as a filter paper, or a non-porous membrane such as growth medium for microorganism growth) supported on the platform.

The body's base section may contain one or more apertures beneath the membrane receiving region in the collection position, e.g. one or more run-off channels, for run-off of the filtered liquid sample. The cartridge may be configured to connect to a vacuum pump for drawing the liquid sample through the capture media and into the one or more run-off channels.

In some embodiments, there may not be a requirement to filter the sample. If the sample does not need filtering, the run-off channels may not be present. Furthermore, the capture medium holder may or may not have an aperture e.g. the platform is a recessed surface which does not have an aperture formed therein.

Following collection and, optionally, filtration of the liquid sample in the collection position of the capture medium holder, and the capture medium it carries, may be moved to the inspection position illustrated in FIG. 1B. In the inspection position, the platform of the capture medium holder is aligned with inspection window 203 of the top section such that the capture medium may be inspected with or without a microscope. As described in more detail below, the base section of the cartridge may comprise a chamber or channel aligned with the capture medium when the capture medium holder is in the inspection position.

In some embodiments an analytical substance, for example, a growth medium e.g. with or without antibiotics, and/or with or without antibodies, may be disposed in the chamber and the chamber may be configured such that the analytical substance contacts the capture medium in the inspection position.

The analytical substance may be brought into contact with the growth medium by any suitable method. In some embodiments, the analytical substance is brought into contact with the growth medium using an ampoule containing the analytical substance as described in more detail below.

It will be understood that the pores of a porous capture medium, e.g. a porous membrane may be selected such that a target microorganism may be collected thereon. Optionally, pore sizes are in the range of about 1 nm to about 100 μm. Optionally, pore sizes are in the range of about 0.2 μm to about 100 μm.

The inlet port may comprise any suitable structure allowing the liquid sample to be introduced into the cartridge body. In some embodiments, the inlet port comprises a fitting mechanism such as, for example, a push-fit mechanism, a Luer fitting or a screw thread for a syringe.

The base section 400 and top section 200 of the cartridge body may be urged together during collection, sample growth and/or inspection by any suitable securing means including, without limitation, screws, nuts, bolts, clamps and cams. The base and top sections of the cartridge body may each comprise one or more threaded or unthreaded apertures 205, 405 extending therethrough. Apertures of the base and top sections may be aligned to receive a screw, a bolt or the like to secure and press together the base and top sections of the cartridge body.

Following collection of a sample on the capture medium, the securing means may be loosened, e.g. such that the top and base sections are still secured together but movable relative to one another, or the securing means may be removed to allow movement of the capture medium holder from the collection position to the inspection position. After movement of the capture medium holder to the inspection position, the securing means may be retightened or reattached.

The capture medium in the inspection position may be analysed by any optical analysis technique known to the skilled person including, without limitation, analysis with a microscope; inspection with the naked eye; and fluorescence measurements using a light source and a photodetector configured to detect light absorbed and re-emitted by a fluorescent marker.

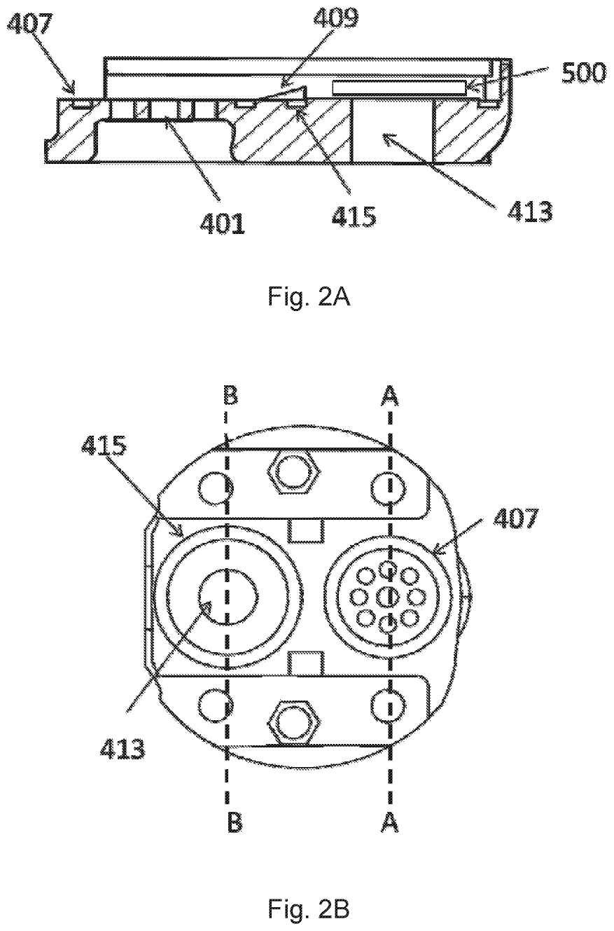

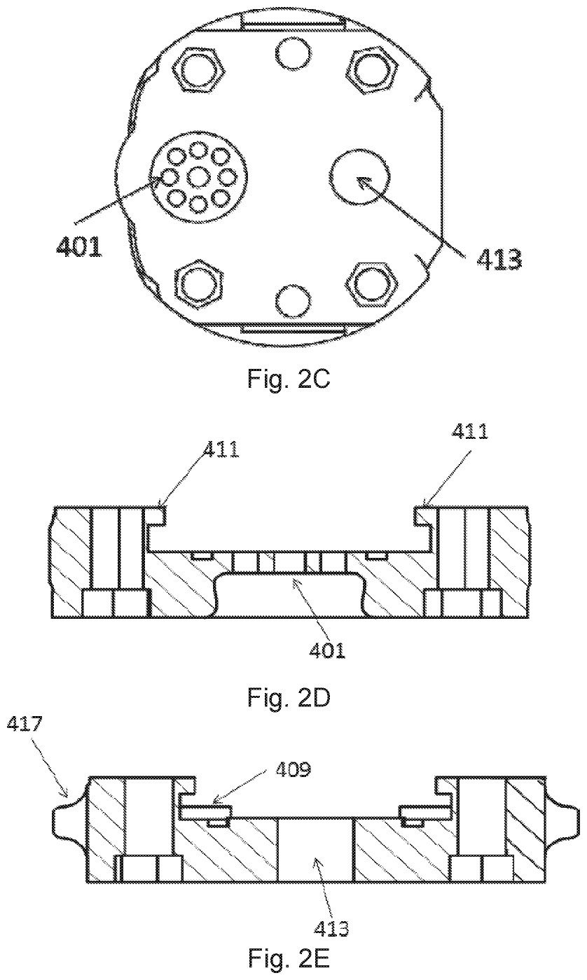

The base section according to some embodiments will now be described in more detail with reference to FIGS. 2A-2E.

A collection region of the base section comprises one or more apertures, e.g. run-off channels 401, which are disposed underneath the capture medium when the capture medium holder is in the collection position to allow run-off of filtered liquid. In use a porous pad (not shown) may be disposed between the capture medium and the run-off channels. The porous pad may provide a flat supporting surface for the capture medium. It will be understood that the run-off channels may or may not be present if the liquid sample is not filtered.

A resilient material may be disposed on the lower surface of the cartridge body cavity in the collection region. A resilient material as referred to anywhere herein may be an elastomer, e.g. rubber. The resilient material may form a perimeter 407 around the capture medium when the capture medium holder is in the collection position. The resilient material may be an O-ring. In use, the base section 400 of the cartridge body and the capture medium holder 300 may be urged together such that the resilient material forms a watertight seal between the base section and the capture medium holder. As described in more detail below, the top section of the cartridge body may comprise a projection in the collection region which presses against the top of the capture medium holder 300, thereby pressing the capture medium holder against the resilient material.

A ramp 409 may be disposed on the lower surface of the cartridge body cavity between the collection region and inspection regions of the cartridge body. The ramp may comprise one or more projections on the lower surface of the cartridge body cavity. The ramp may be configured to contact the bottom surface of the capture medium holder 300 when the capture medium holder is moved between the collection and inspection positions.

When the capture medium holder is moved from the collection position to the inspection position, the ramp may cause the capture medium holder to rise above the lower surface of the cartridge body cavity, thereby preventing the capture medium from coming into contact with lower cavity surface while the capture medium holder slides between the collection and inspection positions. Once the membrane is in the inspection position, the ramp may be configured to engage with the capture medium holder to prevent movement thereof when the base and top sections are engaged.

To facilitate movement of the capture medium holder between the collection position and the inspection position, the force with which the base and top sections of the cartridge body are urged together may be reduced or removed, for example by loosening or removing one or more securing means such as screws, bolts or clamps holding the base and top sections together. If a flexible curtain is joined to the base section and the top section then the possibility of ingress of contaminants from the external environment may be limited or eliminated when the base and top sections are not in direct contact.

The base section of the cartridge body may comprise an elongate recess 411 configured to engage with elongate rails of the capture medium holder to retain the capture medium holder in the cartridge body whilst allowing lateral movement of the capture medium holder relative to the cartridge body.

In use, when the capture medium holder is in the inspection position and the base and top sections of the cartridge body are urged together, there may be a gap between the lower surface of the cartridge body cavity and the capture medium holder. A liquid reservoir 500, for example an absorbent pad, growth medium, nutrient agar or a microfluidic device, may be disposed in this gap such that the liquid reservoir is in direct contact with the capture medium is in the inspection position. In other embodiments, the absorbent pad may be retained by the capture medium holder such that the absorbent pad is in contact with the capture medium and such that the absorbent pad, in use, is disposed underneath the capture medium.

The liquid reservoir may contain an analytical substance, e.g. one or more of a growth medium for growth of a target microorganism; reagents for reacting with the collected sample; dyes; antibodies; and lysing solutions. The liquid reservoir may be in fluid communication with a channel 413 of the base section in the inspection region. In use, the analytical substance may be disposed in the channel. The base section may comprise an inlet (not shown) for introducing the analytical substance into the channel. A liquid may be introduced into the channel by any suitable means. In some embodiments, liquid may be introduced from a liquid delivery device, for example, an ampoule 700 as described in more detail below. An outlet of the liquid delivery device may be integrated into the base of the cartridge body, e.g. a part of a modular ampoule containing an outlet as described below. The collected sample in the inspection region may be brought into contact with different substances at different times as may be desired for analysis of the sample.

FIGS. 2A-2C illustrate a base section having a single channel 413 aligned with the platform in the inspection position. In other embodiments, a plurality of channels are aligned with the platform. The channels may be, for example, microfluidic channels. Different analytical substances may be disposed in, or introduced into, each channel for differential analysis of the collected sample.

A resilient material, e.g. an O-ring, may form a perimeter 415 around the capture medium when the capture medium holder is in the collection position. In use, the base section 400 of the cartridge body and the capture medium holder 300 may be urged together such that the resilient material 415 forms a seal between the base section and the capture medium holder.

The cartridge body may comprise elongate protrusions 417 on opposing external surfaces thereof for introduction of the cartridge into a cartridge holder of apparatus for culturing and/or analysis of the sample, e.g. a microscope or incubator. The cartridge may comprise metal. The cartridge may consist of metal. This may allow the protrusions to support heating the cartridge. The cartridge may comprise internal conductive tracks that facilitate heating. The cartridge may have inductive tracts so that when the cartridge is heated from the outside, the inductive tracts may facilitate heat transfer to the internal of the cartridge. In this way, localised heating in the cartridge may be generated.

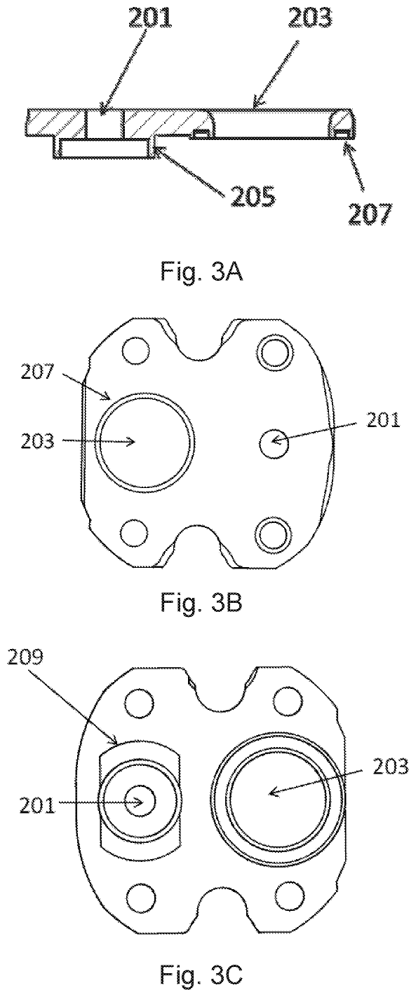

FIGS. 3A-3C schematically illustrate different views of the upper section of the cartridge body.

The collection region of the upper section of the cartridge body comprises an entry port 201 for introduction of fluid into the cartridge body and onto the capture medium when the capture medium holder is in the collection position. The entry port is suitably sealed from the external environment using any means known to the skilled person. It will be appreciated that a sample liquid may be filtered through the capture medium and subsequently drained out of the cartridge body until the pores of the capture medium are blocked, allowing for a large quantity of sample to be collected. In other embodiments, the amount of liquid introduced may be at or below an absorption limit of the capture medium, in which case no filtration may be necessary. In some embodiments, between 1 μl and 1,000 ml of liquid is filtered through the capture medium. In some embodiments, between 1 ml and 1,000 ml of liquid is filtered through the capture medium.

A lower surface of the upper section may comprise a protrusion 205 in the collection region. The protrusion may be urged against the capture medium holder to form a watertight seal between the upper body section and the capture medium holder. As described in detail below, the capture medium holder may comprise a resilient material forming a perimeter around the capture medium, e.g. a rubber washer, and the protrusion 205 may be urged against the resilient material to form the watertight seal. The protrusion may be configured to contact an upper surface of the capture medium holder in an area 209 outside a perimeter defined by the resilient material.

In some embodiments, a sample liquid is introduced through entry port 201 with the capture medium holder in the sample collection position and, following filtration of the sample liquid, the capture medium holder is moved to the inspection position such that the membrane is contacted by one or more analytical substances disposed in channel 413.

In other embodiments, one or more analytical substances are introduced onto the capture medium when the capture medium holder is in the collection position. According to these embodiments, one or more analytical substances may be introduced before or after, preferably after, introduction and filtration of the sample for analysis. The one or more analytical substances may be introduced through entry port 201 or through one or more separate ports (not shown) for introduction of one or more respective analytical substances. The cartridge according to these embodiments may or may not have channel 413. The capture medium holder according to these embodiments may be configured to retain an absorbent pad which, in use, is disposed underneath and in contact with the capture medium. In use, an analytical substance introduced through entry port 201 or through a separate port may pass through the capture medium and be retained in the absorbent pad. In some embodiments, the cartridge comprises a plurality of ports for introduction of different analytical substances and the cartridge is configured to form a plurality of separate chambers in the sample collection position to keep analytical substances introduced from different ports apart from one another. The top section may comprise protrusions defining the plurality of chambers, each chamber being in fluid communication with an entry port for introduction of an analytical substance. The chambers may be microfluidic chambers. The cartridge may also include microfluidic channels. The microfluidic channels may aid the addition of liquid.

In some embodiments, the capture medium holder may be foldable. The foldable capture medium holder may be configured to retain a capture medium and an absorbent pad. Following collection of a sample on the capture medium, the capture medium holder may be folded such that the absorbent pad is disposed underneath and in contact with the capture medium. An analytical substance may be absorbed in the absorbent pad, or may be introduced into the pad through the sample entry port or through a separate port as described above.

The inspection region of the upper section comprises an inspection window 203 to allow inspection of the membrane when the capture medium holder is in the inspection position. The window may be formed from any suitable material, for example glass or transparent plastic. The window may contain one or more holes, for example, air vents, to allow airflow. The window may be a lens. Protrusions may extend from the window which, when brought into contact with the capture medium, define separate compartments for differential analysis.

The upper surface of the body cavity provided by the upper section of the cartridge body may carry a resilient material defining a perimeter 207 around the inspection window, e.g. an O-ring, for forming a seal with the membrane carrier around the capture medium in the inspection position.

FIGS. 4A-4C schematically illustrate a capture medium holder 300 according to some embodiments.

The capture medium holder comprises a drainage aperture 301 through which the sample liquid can pass to the base section following filtration. The perimeter of the drainage aperture may define a platform 303, e.g. a lip, on which the capture medium 600 is supported. The lip may form a bottom part of a groove in the capture medium holder configured to receive a porous membrane. The scope of movement of a porous membrane may be limited when its perimeter is disposed in the groove.

The capture medium holder may comprise first and second parts. The first and second parts of the capture medium holder may lock together in a suitable way. Such an arrangement may allow for easier manufacture. The capture medium holder may be made of multiple parts, each part may comprise or consist of different materials, for example a rubber part to create a seal.

FIGS. 4A-4C illustrate a capture medium holder having only one drainage aperture. In other embodiments, the platform may comprise a plurality of drainage apertures which are covered by the capture medium when the cartridge is in use.

The capture medium holder may comprise a resilient material 305, e.g. a rubber washer, surrounding the drainage aperture and platform. The resilient material may be configured to engage with the protrusion 205 of the top section of the cartridge body to form a seal when the capture medium holder is in the collection position and the top and base sections are urged together.

The capture medium holder may comprise a handle 307 which, in use, extends beyond a perimeter of the cartridge body in at least one of the collection and inspection positions, allowing the capture medium holder to be grasped and moved between the collection and inspection positions.

The capture medium holder may comprise elongate rails 309 on opposing sides thereof. The elongate rails may be configured to engage with the elongate grooves of the cartridge body to allow lateral movement of the capture medium holder relative to the cartridge body.

In some embodiments, the capture medium holder is configured to retain a capture medium and an absorbent pad which, in use, is disposed underneath and in contact with the capture medium. In use, the absorbent pad may absorb analytical substances introduced through the top of the cartridge when the capture medium holder is in the collection position, e.g. through entry port 201 or through another port for introduction of analytical substances, or through one or more channels in the base of the cartridge when the capture medium holder is in the inspection position, e.g. through one or more channels 413.

The cartridge body and the capture medium holder may each independently be made from any suitable material. In some embodiments, the cartridge body is plastic. Preferably, the capture medium holder is plastic or metal. A metal capture medium holder may be sterilised by heating and re-used.

The cartridge may be for single use or multiple use.

The cartridge may be supplied in sterile packaging, with or without a capture medium in the capture medium holder and/or a growth medium in the cartridge body or an external media delivery device.

In some embodiments, the cartridge may be used for analysis of liquids including, without limitation: water, e.g. water intended for use as drinking water, sewage water, coolant fluid, machine lubricant; and human or animal bodily fluids, e.g. blood, urine or saliva.

In some embodiments, the cartridge may be used in detection of bacteria, for example salmonella such as Salmonella Typhi; Campylobacter; Legionella; staphylococcus aureus; Shigella; Vibrio cholerae; E. coli; or antibiotic resistant bacteria such as MRSA, VRE, MDR-TB or CRE.

In some embodiments, the cartridge may be used in detection and/or prevention of diseases associated with bacterial infections, in particular waterborne bacterial diseases such as typhoid, cholera and dysentery; as well as urinary tract infections.

In some embodiments, the cartridge may be used for identifying the presence and/or concentration of an organic or inorganic material in a sample including, without limitation, microplastics and asbestos.

In addition to, or instead of, use of the cartridge for identifying presence and/or concentration of microorganisms in a sample, the capture medium holder may be used in determination of one or more of pH of a liquid sample and the presence and/or concentration of a target analyte in a liquid sample, in particular water. Exemplary target analytes include, without limitation, halide ions, e.g. chloride ions; metal ions, e.g. iron ions; and dissolved oxygen. In this way, the cartridge may be used to determine water quality parameters other than the presence and or concentration of microorganisms in the water.

In some embodiments, a colorimetric disc for measurement of one or more of pH and the presence and/or concentration of a target analyte is contacted with a sample liquid and then inspected through the inspection window of the cartridge with the colorimetric disc disposed in the capture medium holder. The colorimetric disc may be contacted with the sample liquid by disposing the disc in the capture medium holder and introducing the sample liquid into the cartridge body through the inlet port or the colorimetric disc may be contacted with the sample before being placed into the capture medium holder for inspection.

In some embodiments, a colorimetric disc may be disposed within a chamber formed in the cartridge wherein the chamber is in fluid communication with the inlet port, e.g. via a one-way valve. The chamber may be configured to receive the liquid sample, before or after filtration, in order to bring the colorimetric disc into contact with the liquid. The cartridge body may have a window formed therein, separate from the inspection window, allowing inspection of the colorimetric disc.

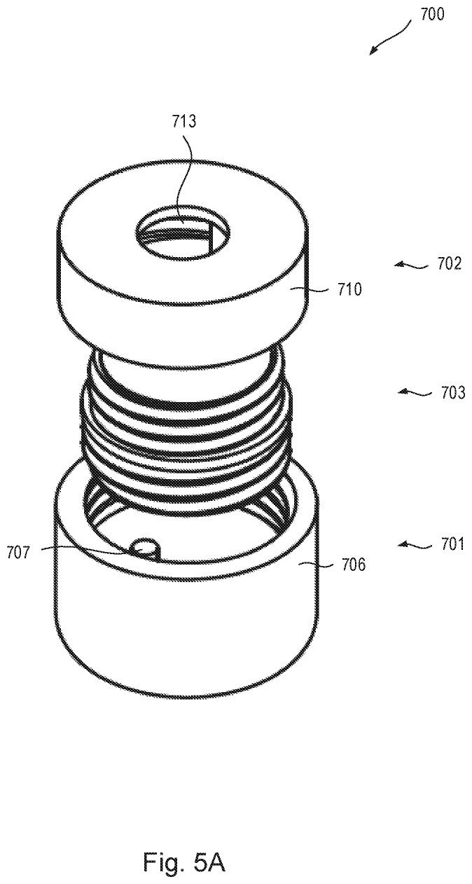

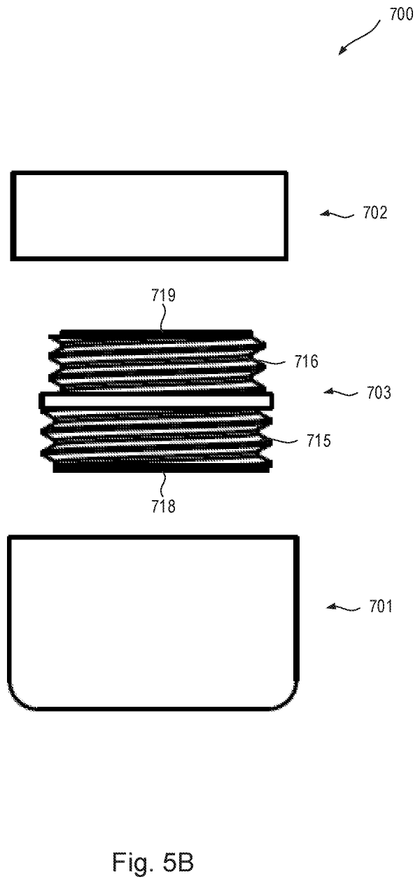

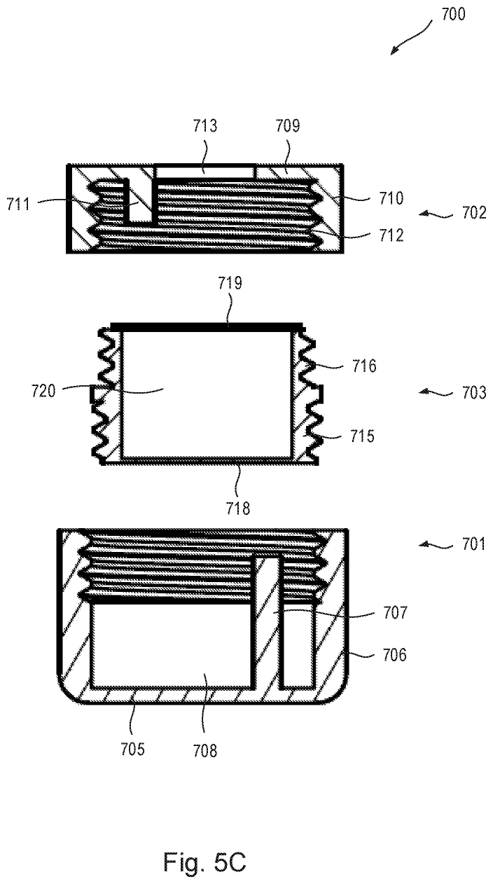

Referring to FIGS. 5A to 5C, in some embodiments, there may be an ampoule 700 for delivery of a liquid, e.g. a growth medium, to the cartridge. The ampoule may contain growth medium. The ampoule 700 may be supplied separately from the cartridge. The ampoule 700 may be used for supply of a substance, for example, a growth medium, to the capture medium holder 300, or a medium embedded in the capture medium holder or ready-mixed in the cartridge. The ampoule 700 may comprise first and second parts 701 and 702 and a connector 703.

The first ampoule part 701 has a body which may have, e.g., a base 705 and a wall 706 defining an internal space 708 in which a first substance, e.g. a solid (optionally a powder) or a liquid may be contained. The first ampoule part illustrated in FIGS. 5A-5C is cylindrical however the body of the first ampoule part may have any shape. The first ampoule part comprises an engagement means, for example, a profile suitable for a push-fitting, a Luer fitting, or an internal female thread. The first ampoule part 701 may have a protrusion 707 protruding from the base 705 into the space 708 inside the first ampoule part. The rod 707 may be parallel to the wall 706.

The second ampoule part 702 has a body with an aperture 713 formed therethrough. The second ampoule part comprises an engagement means, for example, a profile suitable for a push-fitting, a Luer fitting, or an internal female thread. The second ampoule part 702 may have a protrusion 711 extending from an internal surface of the body of the second ampoule part into the space 712 inside the second ampoule part 702.

The connector 703 has a body having first and second sections 715, 716. A first seal 718 extends across an opening of a first section of the connector body and a second seal 719 extends across an opening of a second section of the connector body. An internal volume 720 defined by the body of the connector, the first seal and the second seal may contain a solid, optionally a powder or a liquid. The first and second seals may be frangible. In some embodiments, the first and second seals comprise or consist of a metal foil.

The first section 715 is configured to engage with the first ampoule part 701 and the second section 716 is configured to engage with the second ampoule part. For example, the first and second sections may each have the corresponding surface to allow for a push-fit, screw thread, or counterpart Luer fitting mechanism to the first and second ampoule parts, respectively. The engagement means of the first and second sections may be different. Likewise, the corresponding engagement means of the first and second parts may be different such that the first part cannot engage with the second section of the connector and/or the second part cannot engage with the first section of the connector. The engagement of the first and second parts to the connector may each independently be permanent, or may be reversible. For example, if the cartridge or ampoule is for a single use, there may be no reversible engagement, however, if the cartridge or ampoule parts are intended for a plurality of uses, the engagement of the ampoule parts may be reversible.

In use, a liquid or solid may be disposed in the space 708 of the first ampoule part and the may be disposed in the ampoule may be assembled by engaging the first ampoule part with the first section of the connector and engaging the second ampoule part with the second section of the connector.

Liquids and solids which may be contained in the first part of the ampoule or in the connector include, without limitation, solids selected from dehydrated nutrient media, lyophilized biological enzymes, antibodies, antibiotics, dye, or substrate and liquids selected from sterile water, buffers and dyes. Liquids and solids which may be contained in the first part of the ampoule or in the connector may be sterile.

The first seal 718 is broken by the protrusion 707 upon engagement of the first part with the connector, allowing the substance or substances contained in the connector to mix with the substance or substances contained in the first part.

The second seal 719 is broken by the protrusion 711 upon engagement of the second part with the connector, allowing the mixed substances to escape through ampoule aperture 713. When the cartridge is in the inspection position, the mixed contents of the ampoule may be introduced through aperture 713 of the ampoule, channel 413 of the base of the cartridge and aperture 301 of the capture medium holder onto the capture medium. The ampoule, when in contact with the cartridge, may be inverted to allow mixed substances to flow out of the ampoule aperture 713 and onto the capture medium.

Separation of substances in this way until a sample is ready for analysis may prevent deterioration of the substances. In other embodiments, e.g. where the analytical substance does not readily degrade, the second part may engage directly with a first part having a seal containing an analytical substance in order to release the analytical substance.

Mechanical breakage of the first and second seals by a protrusion has been described above, however it will be understood that any mechanical, physical or chemical means to break the first or second seal may be used, e.g. heat treatment.

In some embodiments, the ampoule is separate from the cartridge.

In some embodiments, at least part of the ampoule is integrated into the cartridge, e.g. the second part is integrated into the cartridge such that the ampoule aperture 713 is aligned with the platform of the capture medium holder in the inspection position.

1. A cartridge comprising a cartridge body and a capture medium holder disposed within the body, the capture medium holder comprising a platform configured to receive a capture medium and the capture medium holder being configured to move relative to the cartridge body between a sample collection position in which the platform is aligned with a sample inlet of the cartridge body and an inspection position in which the platform is aligned with an inspection window of the cartridge body.

2. A cartridge according to claim 1 wherein the cartridge body comprises a top section and a base section which are separable.

3. A cartridge according to claim 2 comprising securing means for releasably urging the top section and base section together.

4. A cartridge according to claim 2 wherein the cartridge is configured to form a watertight seal between the top section of the cartridge body and an upper surface of the capture medium holder in the collection position

5. A cartridge according to claim 2 wherein the cartridge is configured to form an airtight seal between the top section of the cartridge body and an upper surface of the capture medium holder in the inspection position.

6. A cartridge according to claim 2 wherein a ramp is disposed on a surface of the base section between an inspection region and a collection region of the base section wherein the ramp is configured to bias the platform away from the base section surface.

7. A cartridge according to claim 1 wherein the capture medium holder is configured to move laterally relative to the cartridge body between the inspection position and the collection position.

8. A cartridge according to claim 7 wherein the capture medium holder comprises elongate rails configured to engage with elongate grooves within the cartridge body.

9. A cartridge according to claim 1 wherein the platform in the inspection position is configured to be in fluid communication with a chamber of the cartridge body.

10. A cartridge according to claim 1 wherein the capture medium holder is configured to retain an absorbent pad in contact with the capture medium.

11. A cartridge according to claim 9 wherein an analytical substance is disposed in the chamber.

12. A cartridge according to claim 1 sealed in a sterile packaging.

13. A cartridge according to claim 11 wherein the microscope cartridge comprises a capture medium supported on the platform.

14. A cartridge according to claim 1 wherein the capture medium holder is a membrane holder.

15. A kit for forming a cartridge according to claim 1 comprising a cartridge body top section comprising the sample inlet and the inspection window; a cartridge body base configured to form the cartridge body with the cartridge body top section; and the capture medium holder.

16. A kit according to claim 15 further comprising a growth medium.

17. A kit according to claim 15 comprising a capture medium.

18. A kit according to claim 15 sealed in sterile packaging.

19. A method of analysing a liquid sample comprising:
collecting a sample in or on the capture medium comprising introducing the liquid sample into the sample inlet of the cartridge according to claim 1 in which the capture medium holder is in the sample collection position;
moving the capture medium holder to the inspection position; and
analysing the collected sample through the inspection window.

20. (canceled)

21. (canceled)

22. (canceled)

23. (canceled)

24. (canceled)

25. (canceled)

26. An ampoule comprising first part, a second part and a connector wherein:
the connector has an internal volume defined by a body of the connector, a first seal extending across an opening of a first section of the connector body and a second seal extending across an opening of a second section of the connector body;
the first part is configured to engage with the first section of the connector body;
the second part is configured to engage with the second section of the connector body;
the second part has an aperture in a surface thereof;
an internal volume of the first part is in fluid communication with the internal volume of the connector upon engagement of the first part and the connector and breakage of the first seal; and
the aperture of the second part is in fluid communication with the internal volume of the connector upon engagement of the second part and the connector and breakage of the second seal.

27. (canceled)

28. (canceled)
